(19) United States
(12) Patent Application Publication
(10) Pub. No.: US 2026/0328748 A1
(43) Pub. Date: Oct. 22, 2026

(54) Coagulation Prediction for Cheese Manufacturing

(71) Applicant: CHR. HANSEN A/S, Hoersholm (DK)

(72) Inventors: Jannik Torben VINDELOEV, Frederiksberg (DK); Sylvain BROCHERT, Arpajon (FR); Sebastien ROUSTEL, Chalon (FR); Marc FAIVELEY, D'ORNON (FR)

(73) Assignee: CHR. HANSEN A/S, Hoersholm (DK)

(21) Appl. No.: 19/486,121

(22) PCT Filed: May 30, 2024
(86) PCT No.: PCT/EP2024/064905

§ 371 Date: Nov. 20, 2025

(30) Foreign Application Priority Data
May 31, 2023 (EP) 23176372.3

(51) Int. Cl. G01N 33/04

(52) U.S. Cl. CPC G01N 33/04

(57) ABSTRACT

Provided is a computer implemented method for predicting coagulation of milk in a cheese manufacturing process using a coagulant. The method comprises steps of receiving a plurality of predetermined milk coagulation process conditions and inputting the plurality of predetermined milk coagulation process conditions into a model to generate a coagulation state. Furthermore, the method comprises outputting the coagulation state generated by the model, the coagulation state predicting the coagulation of the milk in the cheese manufacturing process over time to determine a curd cutting time.

### TECHNICAL FIELD

The present invention relates to the field of cheese manufacturing. In particular, the present invention relates to a method and system for predicting coagulation of milk in a cheese manufacturing process using coagulant.

### BACKGROUND

For manufacturing cheese, it is often desired to improve the cheese manufacturing process by changing parameters to influence coagulation. Usually, experts are needed which can give advice on what type of coagulant should be used and how to change several other parameters to reach given improvement objectives. The parameters may include parameters with respect to milk characteristics (such as temperature, fat-to-protein ratio, etc.) and/or characteristics of the coagulant.

To do so, a state-of-the-art equipment to measure a coagulation curve is generally used, wherein the coagulation curve may be a firmness progression curve of the milk transforming into curd. The coagulation curve may be measured using a sample of milk from industrial process, wherein similar conditions as the industrial conditions may be reproduced. Many kinds of equipment exist which have been evolved since 1970 (for example, Formagraph, Chymograph, Coagusens from Rheolution), either to measure coagulation of milk samples or to measure the coagulation directly from the milk in process lines.

However, there are several disadvantages when using such state-of-the-art equipment to measure a coagulation curve. For example, the measures are complex, and the number of measures is generally limited because milk samples are needed. Furthermore, one measure usually takes about 40 minutes plus additional time to prepare the measure which limits to, for example, 3 to 4 measures for half a day presence in the factory.

Moreover, some parameter modifications can be difficult to mimic in industrial conditions. For instance, if it is desired to change the milk protein ratio, i.e. the ratio of protein in the milk used for cheese manufacturing, while keeping the remaining parameters in industrial conditions, it may be necessary to prepare large amounts of milk with the changed milk protein ratio which can be used as samples for the measure.

Even though trained experts can often roughly predict the outcome of a simple modification of a process and of parameters, this prediction is only an approximation and the outcome of the modification, such as the coagulation curve, cannot be precisely and accurately predicted. Furthermore, it is impossible for a trained expert to predict the outcome of parameter modifications and the influence on the cheese manufacturing process when several parameters are modified simultaneously.

As indicated above, the state-of-the-art equipment has several disadvantages when measuring the coagulation of milk. However, knowing about the coagulation of the milk is very important when it is desired to manufacture high quality cheese. A method or system that is able to predict or model the coagulation over time with respect to different milk and/or coagulants parameters without the need to rely on the knowledge of experts is missing. There is no method or system that takes general conditions of the milk and/or coagulant into account and predicts a coagulation curve, other coagulation characteristics, and/or process parameters linked to the coagulation, such as a recommended cutting time of the curd, without analyzing a given milk sample.

### SUMMARY

It may be an object of the invention to provide a method and system for accurately and precisely predicting coagulation of milk in a cheese manufacturing process without using milk samples.

According to an aspect, a computer implemented method for predicting coagulation of milk in a cheese manufacturing process using a coagulant may comprise the step of receiving a plurality of predetermined milk coagulation process conditions. Furthermore, the computer implemented method may comprise inputting the plurality of predetermined milk coagulation process conditions into a model to generate a coagulation state and outputting the coagulation state generated by the model. The coagulation state may predict the coagulation of the milk in the cheese manufacturing process over time to determine a curd cutting time.

According to another aspect, a system for predicting coagulation of milk in a cheese manufacturing process using a coagulant may comprise a processing unit. The processing unit may be configured to receive a plurality of predetermined milk coagulation process conditions and input the plurality of predetermined milk coagulation process conditions into a model to generate a coagulation state. Furthermore, the processing unit may be configured to output the coagulation state generated by the model, wherein the coagulation state may predict the coagulation of the milk in the cheese manufacturing process over time to determine a curd cutting time.

### BRIEF DESCRIPTION OF THE DRAWINGS

FIG. 1 shows an embodiment of a computer implemented method for predicting coagulation of milk in a cheese manufacturing process using a coagulant.

FIG. 2 shows an example of a display illustrating a coagulation state predicting the coagulation of milk over time.

FIG. 3 shows an example of a model for predicting the coagulation of milk over time.

FIG. 4 shows an example of determining the curd cutting time based on a predetermined property of the cheese and an outputted coagulation state.

FIG. 5 shows an example of a system for predicting the coagulation of the milk over time.

### DETAILED DESCRIPTION

Some of the embodiments contemplated herein will now be described more fully with reference to the accompanying drawings. Other embodiments, however, are contained within the scope of the subject matter disclosed herein, the disclosed subject matter should not be construed as limited to only the embodiments set forth herein; rather, these embodiments are provided by way of example to convey the scope of the subject matter to those skilled in the art.

Generally, all terms used herein are to be interpreted according to their ordinary meaning in the relevant technical field, unless a different meaning is clearly given and/or is implied from the context in which it is used. All references to a/an/the element, apparatus, component, means, step, etc. are to be interpreted openly as referring to at least one instance of the element, apparatus, component, means, step, etc., unless explicitly stated otherwise. The steps of any methods disclosed herein do not have to be performed in the exact order disclosed, unless a step is explicitly described as following or preceding another step and/or where a step must necessarily follow or precede another step due to some dependency. Any feature of any of the embodiments disclosed herein may be applied to any other embodiment, wherever appropriate. Likewise, any advantage of any of the embodiments may apply to any other embodiments, and vice versa. Other objectives, features, and advantages of the enclosed embodiments will be apparent from the following description.

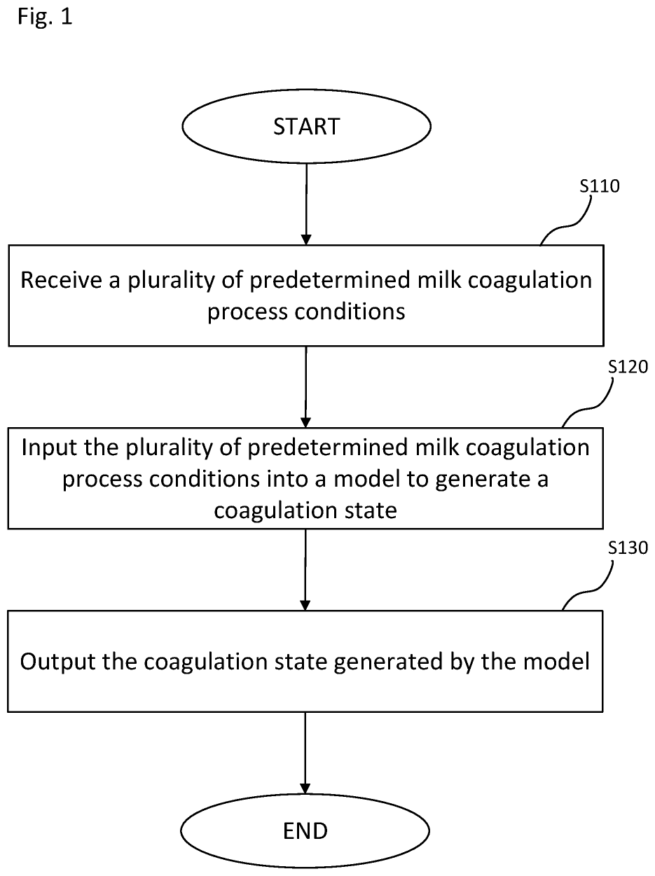

FIG. 1 shows an embodiment of a computer implemented method for predicting or modelling coagulation of milk in a cheese manufacturing process using a coagulant. The coagulation of the milk can be predicted or modelled before the coagulant is actually added to the milk and before the cheese manufacturing is started. Furthermore, the coagulation prediction can be performed without milk samples.

The coagulant may be a compound or agent which is added to the milk to help thicken or solidify the milk for manufacturing cheese. The thickened or solidified milk may be called curd, gel, or coagulum.

Coagulation can occur in different ways. For example, rennet or acid may be used as coagulant to encourage coagulation. Rennet coagulation, i.e. using rennet for encouraging coagulation, may refer to the addition of enzymes to the milk in order to thicken the milk. If acid coagulation is desired, acid is added to the milk to encourage coagulation. Furthermore, heat may be used to influence the coagulation process.

In order to be able to produce high quality cheese, it is often necessary to predict the coagulation of the milk to be used in the cheese manufacturing process over time and determine a curd cutting time with high accuracy. The curd cutting time may indicate a time that has elapsed after the coagulant has been added to the milk and may indicate when to cut the curd for a rennet coagulated cheese or break the curd for an acid coagulated cheese. Breaking the curd may be done by stirring or ladling. Cutting the curd may be done using a curd knife or the like.

The purpose of cutting or breaking the curd may be to increase its surface area to volume ratio and increase its expulsion of whey. This is done to reduce the water content of the curd and in the final cheese.

Usually, the curd cutting time depends on the type of cheese to be manufactured. If, for example, a softer cheese, like camembert or the like, is desired, the curd cutting time, i.e. the time that has elapsed after having added the coagulant to the milk, may be different compared to a case when a harder cheese, like parmesan or the like, is desired. Thus, recipes for manufacturing cheese usually specify a curd cutting time to ensure that the curd is cut or broken when it has the right firmness.

However, the coagulation of the milk and, thus, the curd cutting time, depends highly on the characteristics of the milk and/or the coagulant used. When recipes specify a curd cutting time, the influence of the characteristics of the milk and/or the coagulant on the curd cutting time is neglected, resulting in cheese with low quality. Thus, it is necessary to specify the curd cutting time with respect to the milk and/or coagulant characteristics in order to achieve high quality cheese.

In order to predict the coagulation of the milk over time and, thus, determine the curd cutting time with high accuracy without using milk samples, a computer implemented method as exemplary shown in FIG. 1 is described. The computer implemented method may be a method implemented on a computing device, wherein a processing unit (described in more detail below) of the computing device may execute the method steps. As shown in FIG. 1, the method may comprise the step of receiving (S110) a plurality of predetermined milk coagulation process conditions, i.e. at least two predetermined milk coagulation process conditions. The plurality of predetermined milk coagulation process conditions may be input by a user, such as a cheese manufacturer, or may be received by a device which transmits the plurality of predetermined milk coagulation process conditions to the processing unit executing the method steps.

The predetermined milk coagulation process conditions may indicate the conditions, i.e. characteristics, of the milk and/or coagulant to be used for the cheese manufacturing process. For example, the plurality of predetermined milk coagulation process conditions comprises a plurality of milk and/or coagulant parameters. For instance, the plurality of predetermined milk coagulation process conditions comprises at least two of an International Milk-Clotting Units per gram of protein (IMCU/g) value, a renneting pH value, a temperature value, a value indicating an amount of calcium chloride (CaCl₂), a percentage value of protein, a parameter indicating a fat-to-protein ratio, and a type of coagulant.

The temperature value may indicate the temperature of the milk to be used for manufacturing the cheese, the percentage value of the protein may indicate the amount of protein in the milk, and the fat-to-protein ratio may indicate the ratio of fat to protein in the milk. Thus, these parameters may further define the milk used for manufacturing the cheese.

The value indicating an amount of CaCl₂ may indicate the amount of CaCl₂ to be added to the milk during cheese manufacturing. CaCl₂ is often added to the milk during cheese manufacturing to improve the rennet coagulation process. The value indicating the amount of CaCl₂ may be in parts per million (ppm).

The renneting pH value may indicate the pH value of the rennet used for encouraging the coagulation. If acid is used instead of rennet, the predetermined milk coagulation process condition may comprise characteristics of the acid.

The type of coagulant may indicate whether rennet or acid is used for encouraging the coagulation. Furthermore, the type of coagulant may indicate a specific type of rennet or acid to be used. For example, the type of coagulant may be CHY-MAX Supreme, CHY-MAX M, CHY-MAX, Maxiren, Maxiren XDS or the like to encourage the coagulation of the milk. As shown in FIG. 1, the method may further comprise the step of inputting (S120) the plurality of predetermined milk coagulation process conditions into a model to generate a coagulation state and outputting (S130) the coagulation state generated by the model. The coagulation state may be displayed on a display unit and/or outputted to another unit, such as a curd cutting entity (described later) which automatically cuts the curd depending on the coagulation state.

The coagulation state may predict the coagulation of the milk in the cheese manufacturing process over time to determine a curd cutting time. Thus, it is possible to accurately and quickly determine the coagulation of the milk over time and the curd cutting time. This means that it is possible to manufacture high quality cheese without using milk samples by considering a plurality of milk coagulation process conditions.

According to an example, the coagulation state may be a coagulation curve or firmness procession curve of the milk over time. It may indicate the firmness increase of the milk over time.

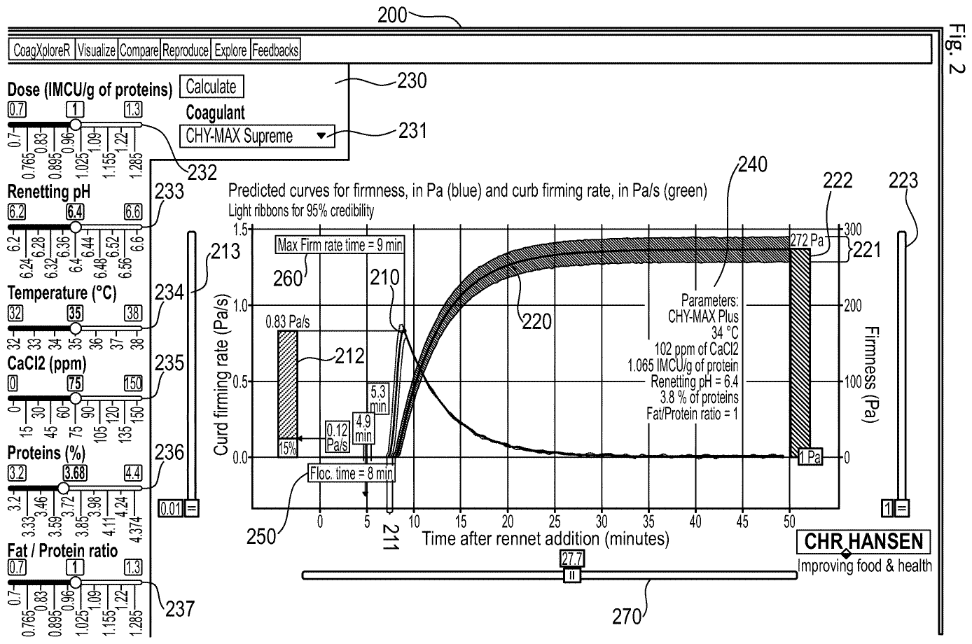

As mentioned above, the coagulation state may be displayed to a user, such as a cheese manufacturer, using a display unit. The display unit may display a graphical user interface (GUI) which is exemplary shown in FIG. 2.

FIG. 2 shows an example of a graphical user interface (GUI) or display 200 illustrating a coagulation state predicting the coagulation of milk over time. The coagulation state may be a graph, for example, a coagulation curve 210 or a firmness progression curve 220 of the milk over time. In FIG. 2, both the coagulation curve 210 and the firmness progression curve 220 are displayed, but this is not limiting, and it is also possible that the GUI 200 only shows one of the coagulation curve 210 and the firmness progression curve 220. The coagulation curve 210 and the firmness progression curve 220 may be displayed in different colors for better distinguishability and visibility. For example, the coagulation curve 210 is shown in green, while the firmness progression curve 220 is shown in blue. This is not limiting, and other colors may be used.

As shown in FIG. 2, the x-axis of the coagulation state may indicate the time after adding the coagulant to the milk. The y-axis may depend on the type of coagulation state. For example, the y-axis indicates the curd firming rate in the unit of pascal/seconds (Pa/s) for the coagulation curve 210 and/or the firmness in the unit of pascal (Pa) for the firmness progression curve 220.

According to an example, it is possible to not only show the curves 210 and 220, but also a ribbon or band around each curve 210, 220. The ribbon or band 211 is around curve 210 and may indicate values for 95% credibility for the curd firming rate. The ribbon or band 221 is around curve 220 and may indicate values for 95% credibility for the firmness.

The predetermined milk coagulation process conditions may be set or input, for example, by a user, in the field 230 of the GUI 200. As exemplary shown in FIG. 2, the type of coagulant can be selected in 231, and the dose, i.e. IMCU/g of proteins, can be set in 232. Furthermore, the renneting pH may be set in 233, the temperature of the milk may be set in 234, the amount of CaCl₂ may be set in 235, the percentage of protein in the milk may be set in 236, and the fat-to-protein ratio of the milk may be set in 237. However, this is not limiting, and more or less parameters may be set in field 230. Furthermore, it is possible to set other parameters in field 230 which indicate the characteristics of the milk and/or coagulant to be used for manufacturing the cheese.

Field 240 indicates specific values set or selected for the parameters listed in field 230. By setting the parameters to specific values, the curves 210 and 220 are automatically and immediately generated. Thus, it is possible to quickly and accurately predict the coagulation of the milk over time depending on the parameters without the need of milk samples. If the parameters are modified, the curves 210 and 220 will be immediately updated to indicate the coagulation of the milk over time with respect to the modified parameters.

The GUI 200 may display further information which may be useful to the cheese manufacturer. For example, the GUI 200 displays the flocculation time 250 which indicates when the flocculation of the milk starts after adding the coagulant. The flocculation depends on the parameters of the milk and/or coagulant. In this example, the flocculation would start eight minutes after adding the coagulant to the milk. The GUI 200 may also display the maximum firmness rate time 260 which is the peak of the curve 210. For this example, the maximum firmness rate time 260 is 9 minutes.

Moreover, the GUI 200 may comprise a sliding bar 223, e.g. a scale widget, to allow the user to set a value within a range of minimum and maximum firmness. Here, the range is from 0 Pa to 272 Pa, see also bar 222. The user can drag a slider along a trough to change the value within the range. In this example, the slider is set to 1 Pa. By setting a value to a specific firmness, the corresponding time after coagulant addition is displayed on the curve 220. Thus, the sliding bar 223 is linked to curve 220 and may be used to obtain the corresponding x-value, here time, to the y-value of firmness set via the sliding bar 223.

For the curve 210, a sliding bar 213 may be provided. The sliding bar 213 is similar to the sliding bar 223 but allows the user to set a value within a range of minimum and maximum curd firming rate. Here, the range is from 0 Pa/s to 0.83 Pa/s, see also bar 212. The user can drag a slider along a trough to change the value within the range. In this example, the slider is set to 0.01 Pa/s. By setting a value to a specific curd firming rate, the corresponding time is displayed on the curve 210. Thus, the sliding bar 213 is linked to curve 210 and may be used to obtain the corresponding x-value, here time, to the y-value of curd firming rate set via the sliding bar 213.

According to an example, the bar 212 may not only show the minimum and maximum curd firming rate but also the curd firming rate at 15% of the maximum curd firming rate. In this example, the curd firming rate at 15% is 0.12 Pa/s. The curd firming rate is often an important parameter in coagulation management and linked processes such as drainage of the curd.

According to an example, the GUI 200 may further comprise a sliding bar 270. Similar to sliding bars 213 and 223, the sliding bar 270 allows the user to set a specific time value. The user can drag a slider along a trough to change the time. In this example, the slider is positioned to set a time of 27.7 minutes. By doing so, it is possible to estimate the firmness of the curd at specific time points.

It is noted that the GUI 200 shown in FIG. 2 is merely an example and is not limiting. Less, more, or other information may be displayed which may be useful for a user who desires to obtain cheese with high quality.

As described above, a model may be used for outputting the coagulation state, for example, the curves 210 and 220 depending on the milk and/or coagulant conditions. The model is now further described below.

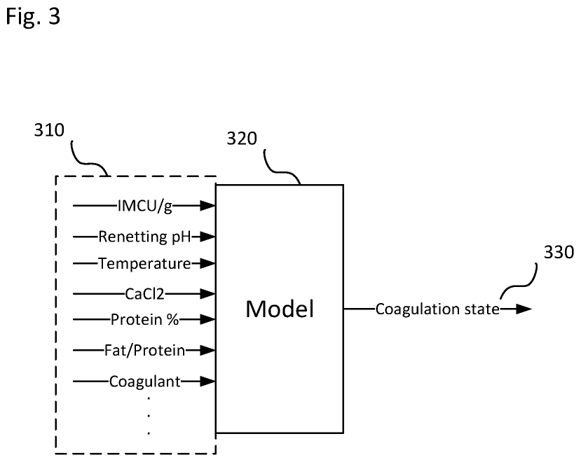

FIG. 3 shows an example of a model 320 for predicting the coagulation of milk over time. The input 310 of the model 320 may comprise a plurality of milk coagulation process conditions, i.e. conditions indicating characteristics of the milk and/or coagulant to be used for manufacturing the cheese. The input 310 may be equal to the parameters set in field 230 of the GUI 200 or may be any other parameters indicating the milk coagulation process conditions. The output 330 of the model 320 may be a coagulation state predicting the coagulation of the milk in the cheese manufacturing process over time. For example, the coagulation state is a coagulation curve or firmness progression curve of the milk over time, as described in more detail above.

The model 320 may be a mathematical model which describes the coagulation of the milk by a set of variables, i.e. parameters indicating the milk coagulation process conditions, and a set of equations that establish relationships between the variables. The model 320 may be set of functions that describe the relations between the different variables in order to output the coagulation state corresponding to the variables. When the variables are changed, the coagulation state output by the model 320 may change, as well. Thus, there is a relationship between the variables input to the model 320 and the coagulation state output by the model 320.

According to an example, the model 320 may be a trained machine learning model, a non-linear regression model, and/or a Bayesian model to generate and output the coagulation state. These examples are not limiting, and any other mathematical model may be used to generate and output the coagulation state.

The Bayesian model is a model using Bayesian statistics. The Bayesian model may be continuously updated over time. For example, the Bayesian model is continuously and regularly updated using new data to improve the accuracy for generating the coagulation state and, thus, for determining the curd cutting time. It may be possible to include new data regularly either from specific studies or from field trials run by experts.

The Bayesian model may be a Bayesian hierarchical model. The Bayesian hierarchical model is a statistical model written in multiple levels, i.e. hierarchical form, that estimates parameters of posterior distribution using the Bayesian model.

According to another embodiment, the model 320 may be a trained machine learning model. For example, the trained machine learning model is a trained neural network for generating and outputting the coagulation state. The trained machine learning model may have been trained on a training set comprising of input-output pairs, wherein the input is the plurality of predetermined milk coagulation process conditions and the output is the coagulation state. During the training with the training set, the machine learning model learns a relationship between the predetermined milk coagulation process conditions and the coagulation state. Thus, the trained machine learning model is able to output an accurate coagulation state for a plurality of predetermined milk coagulation process conditions.

As indicated above, the model 320 may also be a non-linear regression model. The non-linear regression model may use a form of regression analysis in which data is fit to a model and then expressed as a mathematical function. For example, the predetermined milk coagulation process conditions are fit to a model and then expressed as a coagulation state which may be a mathematical function or curve to express the coagulation of the milk over time. The mathematical function may be expressed as follows:

Y=f(X, β)+ϵ,

where X may be a vector of the predetermined milk coagulation process conditions, β may be a vector of k parameters, f(·) may be a regression function, and e may be an error term. The k parameters are unitless and may be used to weight the predetermined milk coagulation process conditions to generate the coagulations state.

In other words, non-linear regression relates the predetermined milk coagulation process conditions in a nonlinear, i.e. curved, relationship and may use, as regression function, logarithmic functions, trigonometric functions, exponential functions, power functions, Lorenz curves, Gaussian functions, and other fitting methods to generate the coagulation state.

Now it is described how the curd cutting time may be determined based on the coagulation state output by the model. The curd cutting time either may be determined by a user from the coagulation state displayed, for example, on a display unit (see FIG. 2 as example) or may be automatically determined by a processing unit executing the method. The curd cutting time may be displayed on a display unit or may be output to another unit, like a curd cutting entity (described above), which uses the curd cutting time to further process the curd for manufacturing the cheese.

For example, the computer implemented method described above with respect to FIG. 1, further comprises a step of receiving a predetermined property of a cheese to be obtained by the cheese manufacturing process and a step of determining the curd cutting time based on the predetermined property of the cheese and the outputted coagulation state. As already indicated above, the curd cutting time may represent a time when to cut or break the curd. By using the coagulation state which has been generated based on the predetermined milk coagulation process conditions, the curd cutting time is automatically and accurately determined.

The predetermined property of the cheese may be a firmness value of the cheese to be obtained and/or a curd firming rate of the cheese to be obtained. If the coagulation state output by the model is a firmness curve, the predetermined property of the cheese may be a predetermined firmness value desired to manufacture a specific type of cheese with high quality. The predetermined property of the cheese may be predetermined by the manufacturer manufacturing the cheese.

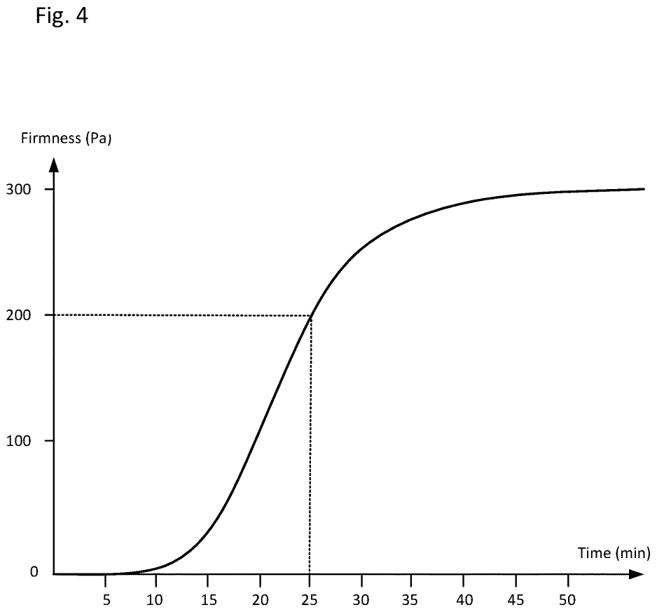

FIG. 4 shows an example of determining the curd cutting time based on a predetermined property of the cheese and an outputted coagulation state. In FIG. 4, an example of a firmness curve is shown, wherein the x-axis represents time in minutes and the y-axis represents firmness in pascal (Pa). The predetermined property may be a firmness value set or determined by a user, such as a cheese manufacturer, and may depend on the type of cheese to be manufactured. In the example shown in FIG. 4, the predetermined property of the cheese may be set to 200 Pa.

By inputting the predetermined property to the processing unit executing the method, the curd cutting time can be automatically determined based on the predetermined property and the coagulation state, here the firmness curve. In this case of a desired firmness of 200 Pa, the processing unit determines the curd cutting time to be 25 minutes (see FIG. 4). Thus, the curd cutting time of 25 minutes indicates that the curd should be broken or cut 25 minutes after having added the coagulant to the milk in order to obtain a high quality cheese with the correct, i.e. desired, firmness.

It is noted that the firmness of 200 Pa is not limiting. Any other firmness value can be set depending on the desired cheese to be manufactured.

According to an embodiment, the processing unit may output an instruction to cut or break the curd at the determined curd cutting time. For example, the determined curd cutting time is output or displayed to a user, such as a cheese manufacturer, so that the user knows when to cut or break the curd. By determining the curd cutting time using the coagulation state output by the model, the curd cutting time is precisely and accurately determined for specific milk and/or coagulant characteristics, because the characteristics of the milk and/or coagulant have been considered. Thus, by outputting a curd cutting time determined as described above, it is ensured that the correct curd cutting time for the actual milk and/or coagulant characteristics is indicated to the user.

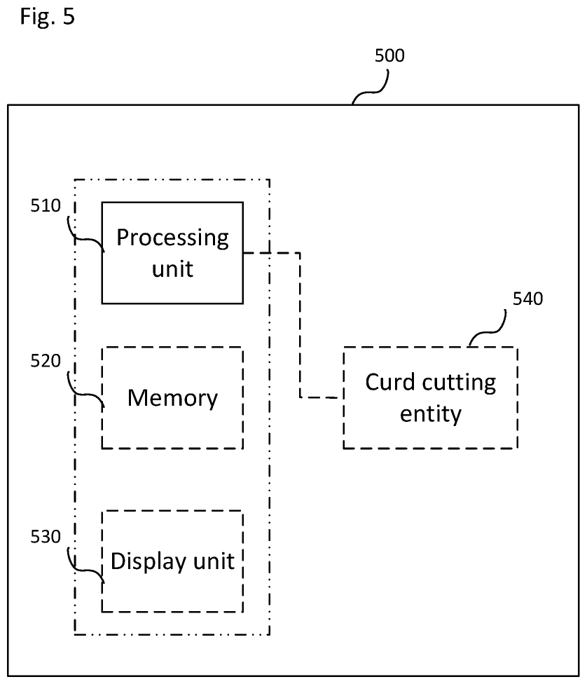

It is also possible that the instruction to cut or break the curd at the predetermined curd cutting time is output to a curd cutting entity, wherein the instruction may indicate the curd cutting time. The curd cutting entity may be a robot or any other entity which receives the instruction and automatically cuts or breaks the curd at the curd cutting time indicated with the instruction. Thus, it is ensured that the curd is automatically cut or broken at the correct curd cutting time and, thus, a high quality cheese is obtained. FIG. 5 shows an example of a system 500 for predicting the coagulation of the milk over time. Optional features or units within the system 500 are indicated by dashed lines. The system 500 may be a computer implemented system and may comprise a processing unit 510. The processing unit 510 may be a processor which executes the method steps described above. The system 500 may further, optionally, comprise a memory 520 for storing the method steps executable by the processing unit 510. Moreover, the memory 520 may store the model which outputs the coagulation state and the coagulation state. Optionally, the system 500 may comprise a display unit 530. The display unit 530 may display a GUI as exemplary shown in FIG. 2. The display unit 530 may comprise means which can be used by the user to input a plurality of predetermined milk coagulation process conditions. Furthermore, the display unit 530 may display the coagulation state output by the model and/or instructions indicating the curd cutting time.

The processing unit 510, the memory 520, and/or the display unit 530 may be part of a computing device (see the dashed box around the processing unit 510, the memory 520, and the display unit 530). The computing device may be a personal computer or the like. However, this is not limiting and, instead of integrating and hardwiring the components in a single computing device, the processing unit 510, the memory 520 and the display unit 530 may be remotely distributed in a network. The network may be cloud-based.

Optionally, the system 500 may comprise a curd cutting entity 540. The curd cutting entity 540 has been described above and may receive an instruction indicating the curd cutting time. According to the received instruction, the curd cutting entity 540 may automatically and accurately cut or break the curd at the curd cutting time.

There is generally considered a computer program product comprising instructions adapted for causing processing and/or control circuitry to carry out and/or control any method described herein, in particular when executed on the processing and/or control circuitry. Also, there is considered a carrier medium arrangement carrying and/or storing a computer program product as described herein.

As shown above, a model, such as a mathematical model, is provided and embedded in a user-friendly application, such as a computer implemented system, so that a user, such as a cheese manufacturer, is able to numerically generate coagulation states for different milk coagulation process conditions. For example, the user can easily and quickly obtain an accurate firmness progression curve of the milk over time and/or an accurate coagulation curve showing the curd firming rate, The curd firming rate may be an important parameter in coagulation management and linked processes such as drainage of the curd. The coagulation states are obtained while saving resources since there is no need for milk samples when predicting the coagulation.

By providing such a model as described above, it is possible to predict the coagulation of milk over time before starting the cheese manufacturing process and actually adding the coagulant to the milk. This allows to explore a lot of possibilities, i.e. a lot of manufacturing conditions and milk coagulation process conditions in a short time and to select milk and coagulant accordingly which should be used in industrial scale for manufacturing cheese.

Furthermore, a reproducible and standardized approach for predicting the coagulation of the milk over time is provided which is independent from experts.

By facilitating the coagulation prediction and increasing the accuracy of the coagulation prediction, cheese can be manufactured with higher quality and less resource waste since no milk samples are needed for coagulation prediction.

It will be apparent to those skilled in the art that various modifications and variations can be made in the entities and methods of this invention as well as in the construction of this invention without departing from the scope or spirit of the invention.

The invention has been described in relation to particular embodiments and examples which are intended in all aspects to be illustrative rather than restrictive. Those skilled in the art will appreciate that many different combinations of hardware, software and/or firmware will be suitable for practicing the present invention.

Moreover, other implementations of the invention will be apparent to those skilled in the art from consideration of the specification and practice of the invention disclosed herein. It is intended that the specification and the examples be considered as exemplary only. To this end, it is to be understood that inventive aspects lie in less than all features of a single foregoing disclosed implementation or configuration. Thus, the true scope and spirit of the invention is indicated by the following claims.

1. A computer implemented method for predicting coagulation of milk in a cheese manufacturing process using a coagulant, said method comprising the steps of:
receiving a plurality of predetermined milk coagulation process conditions;
inputting the plurality of predetermined milk coagulation process conditions into a model to generate a coagulation state; and
outputting the coagulation state generated by the model, the coagulation state predicting the coagulation of the milk in the cheese manufacturing process over time to determine a curd cutting time.

2. The computer implemented method according to claim 1, wherein the plurality of predetermined milk coagulation process conditions comprises a plurality of milk and/or coagulant parameters.

3. The computer implemented method according to claim 1, wherein the plurality of predetermined milk coagulation process conditions comprises at least two of an International Milk-Clotting Units per gram of protein, IMCU/g, value, a renneting pH value, a temperature value, a value indicating an amount of calcium chloride, CaCl2, a percentage value of protein, a parameter indicating a fat-to-protein ratio, and a type of the coagulant.

4. The computer implemented method according to claim 1, wherein the coagulation state is a coagulation curve or firmness progression curve of the milk over time.

5. The computer implemented method according to claim 1, wherein the model is a trained machine learning model, a non-linear regression model, and/or a Bayesian model to generate the coagulation state.

6. The computer implemented method according to claim 4, wherein the Bayesian model is continuously updated over time.

7. The computer implemented method according to claim 5, wherein the Bayesian model is a Bayesian hierarchical model.

8. The computer implemented method according to claim 5, wherein the trained machine learning model is a trained neural network.

9. The computer implemented method according to claim 5, wherein the trained machine learning model has been trained on a training set comprising input-output pairs, wherein the input is the plurality of predetermined milk coagulation process conditions and the output is the coagulation state.

10. The computer implemented method according to claim 1, further comprising:
receiving a predetermined property of a cheese to be obtained by the cheese manufacturing process; and
determining the curd cutting time based on the predetermined property of the cheese and the outputted coagulation state, wherein the curd cutting time represents a time when to cut or break a curd.

11. The computer implemented method according to claim 10, wherein the predetermined property of the cheese is a firmness value of the cheese to be obtained and/or a curd firming rate of the cheese to be obtained.

12. The computer implemented method according to claim 10, further comprising:
outputting an instruction to cut or break the curd at the curd cutting time.

13. A computer program product comprising instructions which, when the program is executed by a computer, cause the computer to carry out the computer implemented method according to claim 1.

14. A system for predicting coagulation of milk in a cheese manufacturing process using a coagulant, said system comprising:
a processing unit configured to:
receive a plurality of predetermined milk coagulation process conditions;
input the plurality of predetermined milk coagulation process conditions into a model to generate a coagulation state; and
output the coagulation state generated by the model, the coagulation state predicting the coagulation of the milk in the cheese manufacturing process over time to determine a curd cutting time.

15-17. (canceled)

18. The system according to claim 14, wherein the model is a trained machine learning model, a non-linear regression model, and/or a Bayesian model to generate the coagulation state.

19-22. (canceled)

23. The system according to claim 14,, wherein the processing unit is further configured to:
receive a predetermined property of a cheese to be obtained by the cheese manufacturing process; and
determine the curd cutting time based on the predetermined property of the cheese and the outputted coagulation state, wherein the curd cutting time represents a time when to cut or break a curd.

24. The system according to claim 23, wherein the predetermined property of the cheese is a firmness value of the cheese to be obtained and/or a curd firming rate of the cheese to be obtained.

25. The system according to claim 23, wherein the processing unit is configured to output an instruction to cut or break the curd at the curd cutting time.

26. The system according to claim 25, further comprising a curd cutting entity, wherein the curd cutting entity is configured to receive the instruction and cut or break the curd at the curd cutting time.
